(19) United States
(12) Patent Application Publication
(10) Pub. No.: US 2024/0050089 A1
(43) Pub. Date: Feb. 15, 2024

(54) SUTURELESS CONNECTOR AND METHODS OF PREPARING AND USING SAME

(71) Applicant: Axogen Corporation, Alachua, FL (US)

(72) Inventors: Kiri K. HAMAKER, Tampa, FL (US); Alec SIMON, Alachua, FL (US); Nikunj Kumar AGRAWAL, Alachua, FL (US); Robert DiLUCCIO, Alachua, FL (US); Angelo SCOPELIANOS, Alachua, FL (US)

(73) Assignee: Axogen Corporation, Alachua, FL (US)

(21) Appl. No.: 18/495,256

(22) Filed: Oct. 26, 2023

Related U.S. Application Data
(63) Continuation-in-part of application No. 18/306,351, filed on Apr. 25, 2023.
(60) Provisional application No. 63/363,648, filed on Apr. 27, 2022.

(51) Int. Cl. A61B 17/06

(52) U.S. Cl. CPC A61B 17/06166; A61B 2017/06176; A61B 2017/00526

(57) ABSTRACT

A tissue connector may include a body formed of one or more layers of material, and at least one barbed suture attached to the body, wherein the barbed suture is formed of a biodegradable material. A method of forming a tissue connector may include forming a body from one or more layers of a material, and attaching at least one barbed suture to the body, wherein the barbed suture is formed of a biodegradable material. A method of repairing tissue using a tissue connector may include orienting the tissue connector around coapted or damaged tissue, so that the coapted or damaged tissue contacts the inner surface of the tissue connector, twisting ends of the at least one barbed suture so that the at least one barbed suture engages the coapted or damaged tissue, and allowing the tissue connector to remain in place, so that the barbed suture degrades over time.

### CROSS-REFERENCE TO RELATED APPLICATIONS

This patent application is a continuation-in-part of U.S. patent application Ser. No. 18/306,351, filed on Apr. 25, 2023, which claims the benefit under 35 U.S.C. § 120 to U.S. Provisional Patent Application No. 63/363,648, filed on Apr. 27, 2022, the entireties of which are incorporated herein by reference.

### TECHNICAL FIELD

Various embodiments of the present disclosure relate generally to tissue connector devices and related methods, and, more particularly, to tissue connector devices having at least one barbed suture, and methods for the preparation and use thereof.

### BACKGROUND

Tissue connectors may be used to do one or more of: connect severed or transected tissue ends at a coaptation site, protect a portion of damaged tissue, protect coapted tissue ends, offload tension at a coaptation site, and/or stabilize coapted or damaged tissue during a tissue healing process. Known tissue connectors may require the use of sutures to attach the tissue connector to tissue being protected and/or repaired. In particular, securing tissue connectors in place at a nerve coaptation site or site of damaged nerve tissue may require microsuturing, by which a surgeon places epineurial sutures through the connector and the tissue. In passing through the tissue, the epineurial sutures may damage the nerve tissue, such as by, for example, disturbing nerve fascicles of a nerve, or other nearby structures, for example, blood vessels. Further, such tissue connectors may be limited in tensile strength.

Therefore, there is a need for a tissue connector that does not require suturing, e.g., microsuturing with epineurial sutures, and has a relatively greater tensile strength. The present disclosure is directed to overcoming one or more of these challenges.

### SUMMARY OF THE DISCLOSURE

According to certain aspects of the disclosure, systems, and methods are disclosed. In one aspect, a tissue connector may comprise a body formed of one or more layers of at least one material, and at least one barbed suture attached to the body so that at least a portion of the at least one barbed suture is exposed along a surface of the body.

In another aspect, a method of forming a tissue connector may comprise forming a body from one or more layers of at least one material, and attaching at least one barbed suture to the body so that at least a portion of the at least one barbed suture is exposed along a surface of the body.

In yet another aspect, a method of repairing tissue using a tissue connector as described herein may comprise orienting the tissue connector around coapted or damaged tissue, so that the coapted or damaged tissue contacts the inner surface of the tissue connector, and twisting ends of the at least one barbed suture so that the at least one barbed suture engages the coapted or damaged tissue.

Additional objects and advantages of the disclosed embodiments will be set forth in part in the description that follows, and in part will be apparent from the description, or may be learned by practice of the disclosed embodiments. The objects and advantages of the disclosed embodiments may be realized and attained by means of the elements and combinations particularly pointed out in the appended claims. An advantage to the disclosed tissue connector devices, and related methods, is that tissue coaptation may be achieved with relative ease of placement and securing of the tissue connector device, without causing damage to the coapted or otherwise damaged tissue or surrounding structures, while providing a tissue connector device having sufficient tensile strength.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 shows a tissue connector, and includes reference to a step of a method of using same, according to one or more embodiments.

FIG. 2 is a perspective view of a barbed suture, including in a tissue connector, according to one or more embodiments.

FIG. 3 shows a tissue connector, and includes details of a configuration of barbed sutures, according to one or more embodiments.

FIG. 4A is a flow chart of a method of forming a tissue connector, according to one or more embodiments.

FIG. 4B is a flow chart of a method of forming a tissue connector, and FIG. 4C is a visual depiction of the method of forming the tissue connector of FIG. 4B, according to one or more embodiments.

FIG. 5 is a flow chart of a method of using a tissue connector, according to one or more embodiments.

FIG. 6 is a graph depicting exemplary suture retention strength of a tissue connector, according to one or more embodiments.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below. However, any terminology intended to be interpreted in any restricted manner will be overtly and specifically defined as such in this Detailed Description section.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a composition, method, or process that comprises a list of elements or steps does not necessarily include only those elements or steps, but may include other elements or steps not expressly listed or inherent to such a composition, method, or process. The relative terms, such as “approximately” and “about,” are generally used to indicate a possible variation of ±10% of a stated or understood value unless indicated otherwise in the specification. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

Tissue connectors that require suturing, e.g., microsuturing, may result in damage to coapted tissue and/or to other nearby structures as a result of the suturing and may have limited tensile strength. The tissue connector and related methods of this disclosure may provide alternatives for tissue coaptation and tissue protection that do not require suturing and may therefore be relatively easier to place and secure at a coaptation site or tissue damage site. Further, the tissue connector and related methods of this disclosure may provide for reliable engagement of tissue, for example, of only an epineurium of a nerve tissue, reducing the risk of damage to the tissue and other structures. In addition, the tissue connector and related methods of this disclosure may provide for a relatively greater tensile strength tissue connector for use at a coaptation site or a site of tissue damage.

Examples of tissue with which the tissue connectors described herein may be used include nerve tissue, such as peripheral nerve tissue or central nervous system tissue. Other types of tissue suitable for the present disclosure include, but are not limited to epithelial tissue, connective tissue, muscular tissue, tendon tissue, ligament tissue, vascular tissue, intestinal tissue, dermal tissue, and cardiac tissue. The tissue may be mammalian tissue, including human tissue and tissue of other primates, rodent tissue, equine tissue, canine tissue, rabbit tissue, porcine tissue, or ovine tissue. In addition, the tissue may be non-mammalian tissue, selected from piscine, amphibian, or insect tissue. The tissue may be allogeneic or xenogeneic to a subject into which the graft is implanted. The tissue may be a synthetic tissue, such as, but not limited to, laboratory-grown or 3D-printed tissue.

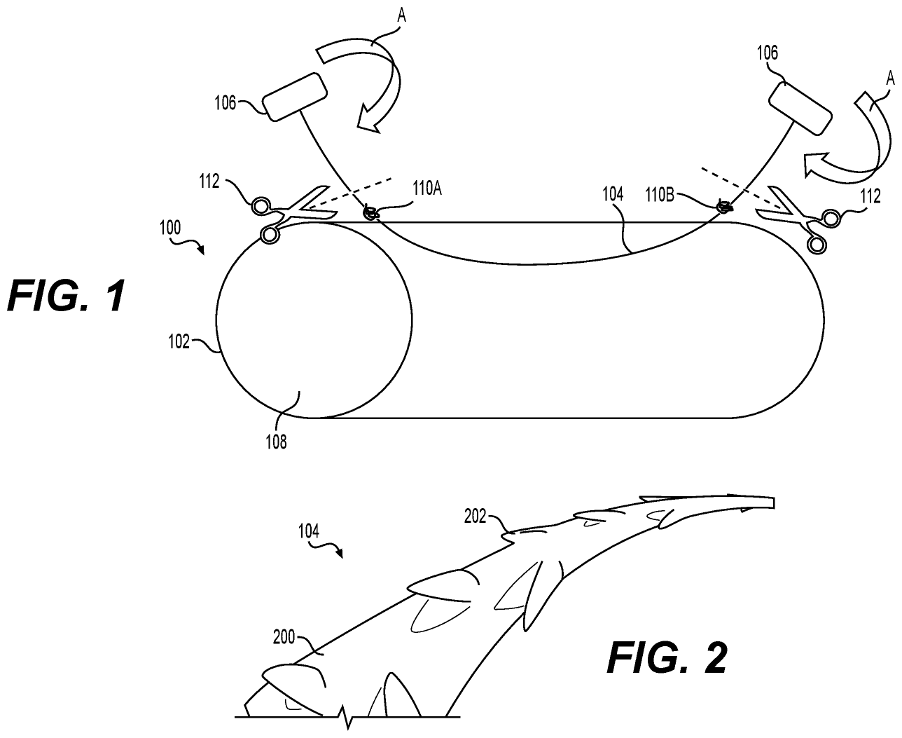

FIG. 1 shows a tissue connector 100 according to one or more embodiments. The tissue connector 100 includes a body 102 formed of one or more layers of material, and at least one barbed suture 104 attached to the body 102. The body 102 may be in the form of a sheet (e.g., a rolled sheet) or a hollow tube and may have any suitable size or shape. In instances in which body 102 may be in the form of a sheet, the sheet may be rolled into a tube prior to attachment to a tissue, or may be wrapped around the tissue for attachment. The one or more layers of material forming body 102 may include one layer up to about 20 layers, such as one layer up to about 12 layers of the material, for example. When two or more layers form the body 102, the layers may be overlapping or aligned. The material used as each of the one or more layers of the body 102 may be small intestinal submucosa (SIS), such as SIS made by Cook Pharmaceuticals, which may be obtained from an animal source, and, in some embodiments, may be obtained from a porcine small intestine. Alternatively, the material may be any flexible material suitable to form a tissue connector. In some aspects, one or more layers of body 102 may be formed of a polymer, e.g., polymer films or a polymer-based material. Suitable polymers may include, e.g., a homo-polymer, copolymer, or blends of two or more polymers (e.g., polyglycolide, polylactide, poly-caprolactone (PCL), polydioxanone (PDO or PDS), polypropylene, polyester polymers), or the like. The polymers may be biodegradable (which includes bioresorbable) and/or biocompatible.

In embodiments of body 102 including multiple layers, each layer may be formed of the same material or from a different material. For example, a polymer layer may be a single layer of which the body 102 is formed or may be one of a plurality of layers. For example, the body 102 may be formed with, or without, the support of another biomaterial layer, formed of, e.g., small intestinal submucosa (SIS) membrane or a natural or synthetic SIS-like membrane. The body 102 may also be comprised of another type of tissue or synthetic membrane like that type of tissue.

In some aspects, a side of body 102 configured to face away from the tissue onto which it is to be applied may be coated with a wet or dried lubricant coating, for example, similar to Axoguard HA+Nerve Protector®, which comprises a coating of hyaluronate and aliginate, as described in U.S. patent application Ser. No. 16/992,857, filed on Aug. 13, 2020, which is incorporated herein by reference in its entirety. In aspects, the lubricant coating or other suitable coating may be configured to inhibit the friction between the membrane and the surrounding tissue, thereby inhibiting the potential for soft tissue attachments. The coating may also comprise one or more compounds that act on the surrounding tissue or on the, e.g., injured tissue to which the tissue connector is applied, such as compounds that encourage tissue healing and/or regeneration, are anti-inflammatory, or are anti-bacterial or anti-microbial or have a wound-healing effect.

The barbed suture(s) 104 may have handles 106 at each end thereof, which may facilitate handling of tissue connector 100. For example, the handles 106 may allow a surgeon (either directly or indirectly via one or more medical devices) to grip both ends of the suture(s) 104 while placing tissue connector 100 relative to tissue. In one embodiment, the suture(s) 104 may be, for example, a Quill® brand barbed suture by Corza Medical. As an example, a barbed suture 104, shown in FIG. 2 in a perspective view, may include a main body 200 with a plurality of barbs 202 protruding from the main body 200. The main body 200 may have a constant diameter, as is shown in FIG. 2, or may have a diameter that changes along its length. The barbs 202 may be arranged in, for example, a longitudinal configuration, in which the barbs 202 are aligned relative to an axis that is parallel to a longitudinal axis of the main body 200, a circumferential configuration, in which the barbs 202 are aligned relative to an circumference of the main body 200, a net configuration, in which the barbs 202 are arranged in a net or a grid pattern on the main body 200, or a spiral configuration, in which the barbs 202 are arranged in a spiral relative to a length of the main body 200 (e.g., the barbs 202 may be arranged in a spiral along the length of the main body 200). In other embodiments, barbs 202 may be spaced irregularly and/or randomly along the main body 200. The suture barbs may be directionally oriented in any suitable way.

In some aspects, barbs 202 and/or main body 200 may be formed of one or more biocompatible and/or biodegradable materials. Barbs 202 and/or main body 200 may be formed of a polymer or a polymer-based material. Suitable polymers may include, e.g., a homo-polymer, copolymer, or blends of two or more polymers (e.g., polyglycolide, polylactide, poly-caprolactone (PCL), polydioxanone (PDO or PDS), polypropylene, polyester polymers), or the like. The polymers may be biodegradable and/or biocompatible. In some aspects, barbs 202 and/or main body 200 may be formed of one or more biodegradable metals or metal alloys, e.g., biodegradable magnesium, zinc, or iron, or alloys of thereof. Main body 200 may be formed of a different material than barbs 202 or may be formed of the same material.

As shown in FIG. 1, the barbed suture(s) 104 may be sewn through the one or more layers of the body 102, so that one or more portions of each suture 104 pass through all of the one of more layers. Alternatively, in a case in which the body 102 is comprised of two or more layers, the barbed suture(s) 104 may be sewn through or arranged relative to at least an innermost layer, so that the suture(s) 104 is embedded or entrapped within or between the layers of the body 102. In such an arrangement, the barbed suture(s) 104 may be exposed, at one or more portions, on an innermost surface 108 of the body 102, and embedded, at alternating portions, between the layers of the body 102. As used herein, the term innermost refers to a surface or layer facing the inner lumen of body 102 that is configured to contact tissue, or, in embodiments in which body 102 is a sheet, the surface of the body 102 that is configured to face to the tissue that tissue connector 100 is to be applied to. Use of tissue connector 100 will be described further below. One or more suture(s) 104 may be arranged roughly parallel to one another on body 102, may be arranged at angles relative to one another, or may be arranged so as to create a mesh-like pattern or hashed pattern on body 102. In some aspects, multiple sutures may be interconnected or woven, or sutures may be spaced apart and may not contact one another.

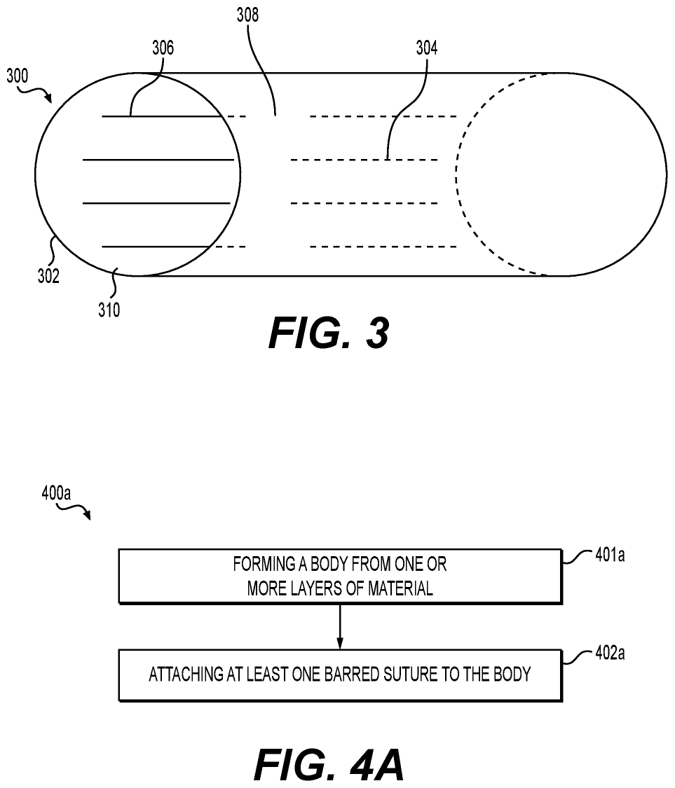

FIG. 3 shows a tissue connector 300 according to another embodiment. Similar to the tissue connector 100 of the embodiment shown in FIG. 1, the tissue connector 300 includes a body 302 formed of a plurality of layers of material, which may be overlapping or aligned. The tissue connector 300 includes one or more barbed sutures 304 attached to the body 302. The body 302 may be in the form of a sheet (e.g., a rolled sheet) or a hollow tube, as discussed in reference to tissue connector 100. The material used as one or more of the layers of the body 302 may be SIS, which may be obtained from an animal source, and, in one embodiment, may be obtained from a porcine small intestine. Alternatively, the material may be any flexible material suitable to form a tissue connector, as discussed above. The multiple layers may be formed of the same material or from one or more different materials. The plurality of barbed sutures 304 may be the same or similar to the sutures 104 described above with respect to FIG. 2, and may be formed of, e.g., the materials discussed above.

In the embodiment of FIG. 3, barbed sutures 304 may be embedded within body 302 such that sections of barbed suture 304 are exposed on an innermost surface 310 of the body 302, and alternating sections of barbed sutures 304 are buried within the multilayered body 302. FIG. 3 depicts an exposed portion 306 of barbed suture 304 along the innermost surface 310 and an embedded portion 308 of barbed suture 304 (e.g., notice the gap where the suture cannot be seen along the innermost surface 310). Embedded portion 308 may be embedded within the innermost layer, within a layer oriented radially outward of the innermost layer, or between layers of body 302. Although FIG. 3 depicts a plurality of barbed sutures 304 arranged longitudinally along a length of body 302, one or more barbed sutures 304 may be arranged in any suitable configuration, e.g., circumferential, net, grid, spiral, one or more independent or interconnected linear configurations, etc., with portions of barbed sutures 304 embedded and portions of barbed sutures 304 exposed on an innermost surface. One or more barbed sutures 304 may be embedded in a serpentine configuration, may be configured to extend in one direction and then turn and extend in the opposite direct, or may have an irregular configuration, for example. As discussed above, one or more suture(s) 304 may be arranged roughly parallel to one another on body 302, may be arranged at angles relative to one another, or may be arranged so as to create a mesh-like pattern or hashed pattern on body 302. In some aspects, multiple sutures may be interconnected or woven, or sutures may be spaced apart and may not contact one another.

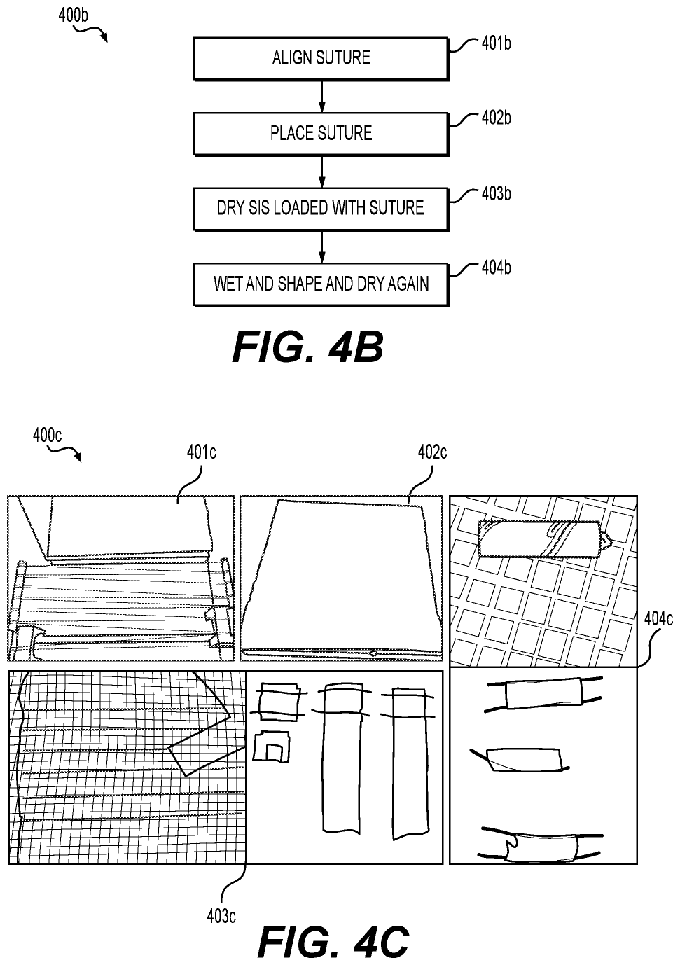

A method 400a of forming a tissue connector according to one or more embodiments is shown and described with reference to FIG. 4A. In particular, FIG. 4A shows the method 400a of forming a tissue connector, such as the tissue connector 100 shown in FIG. 1, or the tissue connector 300 shown in FIG. 3. The method 400a may include the step 401a of forming a body from one or more layers of material, and another or next step 402a of attaching at least one barbed suture to the body. The material of the one or more layers that form the body may be small intestinal submucosa (SIS), which may be obtained from an animal source, for example, from a porcine small intestine. Alternatively, the material may be any flexible material suitable to form a tissue connector, or multiple materials, as described above.

The step 402a of attaching the suture(s) may include sewing each suture into the body. Sewing the sutures may include inserting each suture through all of the one more layers of the body, for example, first, from an outer surface to an inner surface near one end of the body, and then from the inner surface to the outer surface near another end of the body, as shown in FIG. 1. The body may be in the form of a sheet when this step is performed, or may be a tube. This step 402a may further include tying a knot (e.g., knots 110A, 110B in FIG. 1) at at least one end of each suture. In one embodiment, knots 110A, 110B may be tied at both ends of each suture, as shown in FIG. 1. In some aspects, a first knot 110A may be tied at one end of each suture 104, for example, before or as the suture 104 is sewn into the body 102. Moreover, a final or second knot 110B may be tied at another end of the suture 104, for example, after the suture is sewn into the body 102. Knots 110A, 110B may help to secure suture 104 relative to body 102. A medical professional may perform step 402a prior to use of the tissue connector on a subject, or step 402a may be performed by a manufacturer during production of the tissue connector. In some embodiments, step 402a may include attaching handles (e.g., handles 106, as shown in FIG. 1) at ends of each suture to facilitate handling by a user.

In an embodiment in which the tissue connector may have two or more layers, the step 402a of attaching the suture(s) may include sewing each suture into the body. Sewing the sutures may include inserting each suture, e.g., first, from an inner surface of an innermost layer of the body, through or into the innermost layer to embed the suture within one of the layers of the body or between adjacent layers of the body, at one or more locations along a length of the body, and then, back through the innermost layer to the inner surface of the body at alternating locations along the length of the body. The body may be in the form of a sheet when this step is performed, or may be a tube. This step 402a may further include tying a knot at at least one end of each suture. In one embodiment, knots (e.g., knots 110A, 110B in FIG. 1) may be tied at both ends of each suture. A medical professional may perform step 402a prior to use of the tissue connector on a subject, or step 402 may be performed by a manufacturer during production of the tissue connector. Although not shown in FIG. 3, one or more handles (e.g., handles 106, as shown in FIG. 1) may also be attached to one or more of body 302 or sutures 304.

FIG. 4B illustrates various steps of a method 400b of forming the body, and FIG. 4C illustrates the status 400c of the body in each of the various steps. In one or more embodiments, method 400b may include a step 401b of forming the body, which may include aligning barbed sutures or layers of the material, as shown in status 401c of FIG. 4C. For example, as described above, layers of material may be aligned or overlapped in a variety of arrangements. The barbed sutures may be aligned in different patterns, depending on the intended arrangement of the barbed sutures within the material. Further, in some embodiments, step 401b may include aligning the barbed sutures and the layers relative to one another. Method 400b may also include a step 402b of placing suture(s) within the material, as shown in status 402c in FIG. 4C. The sutures may be sewn, for example, within the material in the various arrangements described herein. The sutures may be inserted into the body while the material is dry. Method 400b may also include a step 403b of drying the material once or after the barbed sutures have been placed into the material, as shown in status 403c in FIG. 4C. Additionally, method 400b may include an optional step 404b of wetting the layers of material to shape the tissue connector and then drying again, as shown in status 404c in FIG. 4C. In step 404b, wetting the layers of material to shape the tissue connector may include rolling the layers of material into a tube shape, for example, while the layers of material are wet. If a tissue connector is formed as a sheet in some embodiments, then the shaping step 404b may not be performed. In other aspects, the shaping step 404b may include reshaping or otherwise modifying the shape of the tissue connector. In some aspects, barbed sutures may be sewn into at least an innermost layer of material, and, after attaching the barbed sutures, additional layers of material may be added to one side of the innermost layer of material to form outer layers of material. In such an embodiment, after inserting each barbed suture, the innermost layer of material may be allowed to dry before additional layers of material are added. In other aspects, additional layers of material may be added while the innermost layer of material is wet.

As noted herein, a tissue connector formed in accordance with one of the methods 400a, 400b, and 400c described above may be formed in a wrapped configuration or as a flat sheet. In a case in which the tissue connector is a nerve tissue connector in a wrapped configuration, a diameter of the tissue connector may be a value in a range of about 0.5 mm to about 10 mm, and a length of the tissue connector may be a value in a range of about 5 mm to about 20 mm, for example. Exemplary sizes of nerve tissue connectors may include: 1.5 mm×10 mm, 2 mm×10 mm, 3 mm×10 mm, 4 mm×10 mm, 5 mm×10 mm, 6 mm×10 mm, 7 mm×10 mm, 1.5 mm×15 mm, 2 mm×15 mm, 3 mm×15 mm, 4 mm×15 mm, 5 mm×15 mm, 6 mm×15 mm, and 7 mm×15 mm, wherein the sizes are expressed as diameter×length. And, in a case in which the tissue connector is a nerve tissue connector in a flat sheet configuration, a width of the tissue connector may be a value in a range of about 1 mm to about 15 mm, and a length of the tissue connector may be a value in a range of about 10 mm to about 50 mm, for example. Exemplary sizes of nerve tissue connectors may include: 2 mm×20 mm, 3.5 mm×20 mm, 5 mm×20 mm, 7 mm×20 mm, 10 mm×20 mm, 3.5 mm×40 mm, 5 mm×40 mm, 7 mm×40 mm, and 10 mm×40 mm, wherein the sizes are expressed as diameter×length. In addition, the size of the tissue connector, whether in a wrapped configuration or in a flat sheet configuration, may be determined based on the type of tissue to which the tissue connector is to be attached.

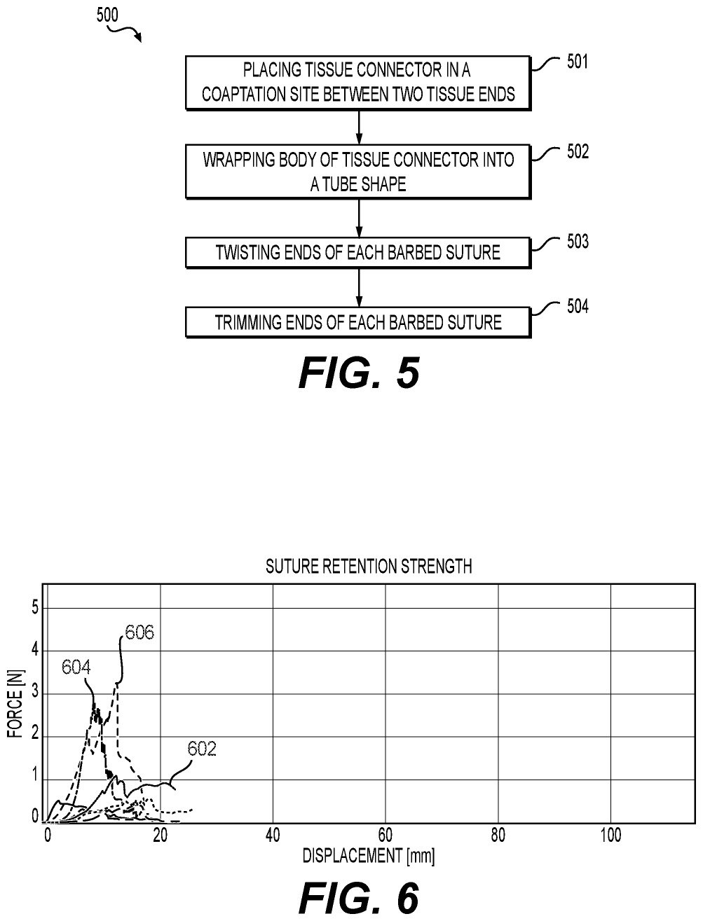

A method 500 of repairing tissue using a tissue connector comprising one or more barbed sutures, according to any one of the embodiments disclosed herein, is shown and described with reference to FIG. 5. In particular, the method 500 may include a step 501 of orienting the tissue connector at a coaptation or repair site adjacent two tissue ends or adjacent damaged tissue. Method 500 may further include a step 502 of wrapping the body of the tissue connector into a tube shape, if the tissue connector is not already shaped as a tube. For example, the tube shape may form a generally cylindrical shape, with open top and bottom ends. Method 500 may also include a step 503 of twisting one or more ends of the barbed suture(s), and a step 504 of trimming or cutting the ends of the suture(s). In the step 501 of orienting the tissue connector at the coaptation or repair site, the tissue connector may be in the form of a sheet, formed of the one or more layers of material. At optional step 502, the body may be wrapped into a tube shape, for example, so that the tissue connector may entubulate or otherwise surround the two tissue ends in the case of severed tissue or otherwise surround the damaged tissue in the case of damaged but not severed tissue. The body may be wrapped akin to a burrito or a taco, for example, to form a generally tubular or open-cylindrical shape. For example, lateral edges of the body may at least partially overlap to form the tubular or open-cylindrical shape. Step 502 may not be performed if the body is already in the form of a tube.

In the step 503, the ends of the suture(s) may be twisted in the same direction (for example, both ends may be twisted in a clockwise direction, or both ends may be twisted in a counterclockwise direction). For example, as shown in FIG. 1, the ends of suture(s) 104 may be twisted in direction A. The respective ends of suture(s) 104 may be twisted by twisting handles 106 coupled to ends of suture(s) 104, as shown in FIG. 1. Alternatively, in some aspects, the ends of the suture(s) may be twisted in opposite directions (for example, one end may be twisted in the clockwise direction, and one end may be twisted in the counterclockwise direction). Twisting the ends of the suture(s) may cause the barbs of the suture(s) to at least partially engage or entangle a surface of the tissue, attaching the tissue connector to the tissue. For example, in the case of nerve tissue, twisting the ends of the suture(s) may cause the barbs of the suture(s) to at least partially engage or entangle in nerve epineurium or outer connective tissue to attach the tissue connector to the nerve tissue. If the barbs are formed of a biodegradable material, e.g., a biodegradable metal alloy, a biodegradable metal, or a biodegradable polymer, then the engagement of the barbs with the tissue may be temporary, and over time, the barbs may degrade, ensuring that barbs are not left permanently in or engaged with the tissue. The ends of the suture(s) may have handles, such as the handles 106 of the embodiment shown in FIG. 1, providing the surgeon or medical professional with a grip on the suture(s) to facilitate twisting. As a result of the step 503 of twisting the ends of the suture(s), the tissue connector may be at least partially secured to the tissue. In the case of coapted tissue, ends of severed tissue, e.g., severed nerve tissue, may be held in place relative to one another within the tissue connector. After the step 503 is performed, an optional step of tying a securing knot in one or both ends of the suture(s) may be performed. For example, step 503 may include tying knot 110B. In addition, in step 504, the free ends of the suture(s) may optionally be trimmed, for example, using one or more trimming devices 112 (e.g., scissors), as shown in FIG. 1, relatively close to ends of the suture(s) that protrude from the tissue connector. In the embodiment shown in FIG. 1, the tissue connector is trimmed at a location between a knot (e.g., knots 110A, 110B) and a handle (e.g., handles 106) at each end of the suture(s). The resultant sutureless connector may be fixed to tissue, e.g., nerve ends, with strength equivalent to or greater than 8-0 sutures.

FIG. 6 is a graph depicting exemplary retention strength against displacement amount of a tissue connector according to one or more embodiments. More specifically, a tissue connector according to the embodiments described herein may provide a relatively greater retention strength as compared to that of known connectors that use epineurial sutures. In retention tests have been performed using ASTM suture retention tests on tissue connectors, according to one of the embodiments described herein. FIG. 6 depicts the results of these retention tests, and, in particular, shows that exemplary tissue connectors of this disclosure had relatively greater retention strength, as they endured greater force before ends thereof became displaced. In particular, tissue connector prototypes according to embodiments described herein demonstrated a retention strength of about 0.5 N to about 3 N, as shown in at least lines 602, 604, and 606 in the graph of FIG. 6, which correlate to the three lines that achieve the highest measures of suture retention strength in terms of force in FIG. 6.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A tissue connector, comprising:
a body formed of one or more layers of at least one material; and
at least one barbed suture attached to the body so that at least a portion of the at least one barbed suture is exposed along a surface of the body, wherein the barbed suture is formed of a biodegradable material.

2. The tissue connector of claim 1, wherein the barbed suture is formed of a biodegradable polymer.

3. The tissue connector of claim 2, wherein the biodegradable polymer includes one or more of polyglycolide, polylactide, poly-caprolactone (PCL), polydioxanone (PDO or PDS), polypropylene, or a polyester polymer.

4. The tissue connector of claim 1, wherein the barbed suture is formed of a biodegradable metal or a biodegradable metal alloy.

5. The tissue connector of claim 4, wherein the biodegradable metal or the biodegradable metal alloy includes one or more of magnesium, zinc, or iron.

6. The tissue connector of claim 1, wherein the barbed suture comprises a plurality of barbs and a main body, wherein the plurality of barbs and the main body are formed of the same material.

7. The tissue connector of claim 1, wherein the barbed suture comprises a plurality of barbs and a main body, wherein the plurality of barbs and the main body are formed of different materials.

8. The tissue connector of claim 1, wherein the body is a hollow tube, and wherein the surface is an innermost surface of the hollow tube.

9. The tissue connector of claim 1, wherein the body is a sheet.

10. The tissue connector of claim 1, wherein the at least one material includes at least one of small intestinal submucosa (SIS) or a biodegradable polymer.

11. The tissue connector of claim 1, wherein the at least one barbed suture is sewn through the one or more layers that form the body so that portions of the at least one barbed suture pass through all of the one or more layers.

12. The tissue connector of claim 1, wherein the one or more layers includes two or more layers, and wherein the at least one barbed suture is sewn within or between layers, of the two or more layers, so that portions of the at least one barbed suture are entrapped within or between the two or more layers.

13. The tissue connector of claim 1, wherein the body is a hollow tube, the one or more layers includes two or more layers, and the at least one barbed suture is sewn into the body with portions of the at least one barbed suture being exposed on an innermost surface of the hollow tube, and alternating portions of the at least one barbed suture being buried within the two or more layers.

14. The tissue connector of claim 1, wherein the one or more layers includes 1 layer to about 12 layers.

15. The tissue connector of claim 1, wherein the at least one barbed suture is oriented, relative to the body of the tissue connector, in one of:
a longitudinal configuration;
a circumferential configuration;
a net configuration; and
a spiral configuration.

16. A method of forming a tissue connector, the method comprising:
forming a body from one or more layers of at least one material; and
attaching at least one barbed suture to the body so that at least a portion of the at least one barbed suture is exposed along a surface of the body, wherein the at least one barbed suture is formed of a biodegradable material.

17. The method of claim 16, wherein the at least one barbed suture is formed of a biodegradable polymer.

18. The method of claim 17, wherein the biodegradable polymer includes one or more of polyglycolide, polylactide, poly-caprolactone (PCL), polydioxanone (PDO or PDS), polypropylene, or a polyester polymer.

19. The method of claim 16, wherein the at least one barbed suture is formed of a biodegradable metal or a biodegradable metal alloy.

20. The method of claim 19, wherein the biodegradable metal or the biodegradable metal alloy includes one or more of magnesium, zinc, or iron.

21. The method of claim 16, wherein each of the at least one barbed sutures comprises a plurality of barbs and a main body, wherein the plurality of barbs and the main body are formed of the same material.

22. The method of claim 16, wherein each of the at least one barbed sutures comprises a plurality of barbs and a main body, wherein the plurality of barbs and the main body are formed of different materials.

23. The method of claim 16, wherein the at least one material includes at least one of small intestinal submucosa (SIS) or a biodegradable polymer.

24. The method of claim 16, wherein the body is a sheet.

25. The method of claim 16, wherein the attaching the at least one barbed suture includes:
inserting each suture, of the at least one barbed suture, through all of the one or more layers of the body, (a) from an outer surface to an inner surface near one end of the body, and (b) from the inner surface to the outer surface near another end of the body; and
tying a knot at at least one of an end of each suture.

26. The method of claim 25, wherein the forming the body includes:
providing the one or more layers in a wet state; and
allowing the one or more layers to dry,
wherein the inserting of each suture is performed after the one or more layers have dried.

27. The method of claim 16, wherein the one or more layers includes two or more layers, and the attaching the at least one barbed suture includes:
inserting each suture, of the at least one barbed suture, (a) from an inner surface through an innermost layer, of the two or more layers of the body, at one or more locations along a length of the body, and (b) back through the innermost layer to the inner surface at alternating locations along the length of the body.

28. The method of claim 27, wherein attaching at least one barbed suture to the body includes inserting each suture while the innermost layer of material is in a wet state, and wherein the method further comprises:
allowing, after the inserting of each suture, the innermost layer of material to dry; and
adding one or more additional layers of material on an outer surface of the dried innermost layer.

29. The method of claim 16, wherein the forming the body includes:
aligning layers of the material; and
rolling the layers of material into a tube shape.

30. The method of claim 16, wherein the one or more layers includes 1 layer to about 12 layers.

31. The method of claim 16, wherein the at least one barbed suture is oriented, relative to the body of the tissue connector, in one of:
a longitudinal configuration;
a circumferential configuration;
a net configuration; and
a spiral configuration.

32. A method of repairing tissue using a tissue connector according to claim 1, the method comprising:
orienting the tissue connector around coapted or damaged tissue, so that the coapted or damaged tissue contacts an inner surface of the tissue connector;
twisting ends of the at least one barbed suture so that the at least one barbed suture engages the coapted or damaged tissue; and
allowing the tissue connector to remain in place, so that the barbed suture degrades over time.

33. The method of claim 32, wherein the biodegradable material is a biodegradable polymer.

34. The method of claim 32, wherein the biodegradable material is a biodegradable metal or a biodegradable metal alloy.

35. A tissue connector, comprising:
a body formed of one or more layers of at least one material; and
at least one barbed suture attached to the body so that at least a portion of the at least one barbed suture is exposed along a surface of the body, wherein the barbed suture is formed of a polymer.
